(19) United States
(12) Patent Application Publication
(10) Pub. No.: US 2021/0217325 A1
(43) Pub. Date: Jul. 15, 2021

(54) SYSTEM OF IMPROVING NEUROPLASTICITY AND RECOVERY DURING OCCUPATIONAL, PHYSICAL, AND RELATED THERAPIES

(71) Applicant: ShadowDraw Inc., Mount Carroll, IL (US)

(72) Inventors: Nicklas Pernblad, Göteborg (SE); Abby Anderson, Mount Carroll, IL (US); Martin McDonough, Mount Carroll, IL (US)

(21) Appl. No.: 17/213,073

(22) Filed: Mar. 25, 2021

Related U.S. Application Data
(63) Continuation-in-part of application No. 15/942,144, filed on Mar. 30, 2018.
(60) Provisional application No. 62/479,094, filed on Mar. 30, 2017.

(51) Int. Cl. G09B 19/00; G06F 3/041; G06F 3/0354; G06F 3/0488; G09B 11/00; G09B 5/02; A61B 5/11

(52) U.S. Cl. CPC G09B 19/003; G06F 3/041; G06F 3/03545; A61B 2505/09; G09B 11/00; G09B 5/02; A61B 5/1124; G06F 3/04883

(57) ABSTRACT

The present invention relates generally to a system for rehabilitating a patient. The system including a computer, a touch screen that is coupled to the computer, and a stylus that is coupled to the computer and configured for use with the touch screen. Stored within the computer is a tutorial that is configured to prompt the patient to redraw an object that is displayed on the touch screen using the stylus in combination with the touch screen. The object is subdivided into a plurality of portions, the tutorial includes a sequence of steps that are to be performed by the patient. In each of the steps of the sequence of steps, the patient is prompted to redraw at least one portion of the object.

### CROSS-REFERENCE TO RELATED APPLICATIONS

This application is a continuation-in-part of pending U.S. patent application Ser. No. 15/942,144, entitled “System of Improving Neuroplasticity and Recovery During Occupational, Physical, and Related Therapies,” filed Mar. 30, 2018, which is incorporated by reference herein in its entirety, and which claims priority to U.S. Provisional Patent Application Ser. No. 62/479,094, entitled “ShadowDraw: A Drawing and Redrawing Software, Technology and Marketplace,” filed Mar. 30, 2017, which is incorporated by reference herein in its entirety. Accordingly, priority is claimed under 35 U.S.C. § 120 to U.S. patent application Ser. No. 15/942,144, and under 35 U.S.C. § 119(e) to U.S. Provisional Patent Application No. 62/479,094, and the content of both U.S. patent application Ser. No. 15/942,144 and U.S. Provisional Patent Application No. 62/479,094 are incorporated by reference as thought fully set forth herein.

### FIELD OF THE INVENTION

The present invention is related generally to a system for improving neuroplasticity and recovery during occupational and physical therapy, such as with stroke, traumatic brain injury, and other conditions requiring rehabilitation and therapy.

### BACKGROUND

Techniques of occupational therapy were used as early as 100 BCE by Greek physician Asclepiades, who treated patients with a mental illness humanely using therapeutic baths, massage, exercise, and music. Three hundred years later in the Roman era, Celsus prescribed music, travel, conversation and exercise to his patients. Unfortunately, by medieval times the use of these interventions with people with mental illness disappeared in Europe.

Some physicians in 18th-century Europe, such as Philippe Pinel and Johann Christian Reil, switched from the use of metal chains and restraints to rigorous work and leisure activities for their patients, with positive results. The emergence of occupational therapy challenged the views of mainstream scientific medicine. By the early 1900's, the health profession of occupational therapy was conceived.

Instead of focusing purely on the medical model, occupational therapists argued that a complex combination of social, economic, and biological reasons cause dysfunction. Principles and techniques were borrowed from many disciplines—including but not limited to physical therapy, nursing, psychiatry, rehabilitation, self-help, orthopedics, and social work. Early professionals merged scientific and medical principles with highly valued ideals, such as having a strong work ethic and the importance of crafting with one's own hands.

Physical therapy is used to improve a patient's quality of life through examination, diagnosis, prognosis, and physical intervention. It works towards addressing any illnesses or injuries that limit a person's abilities to move and perform functional activities in their daily lives. Physicians like Hippocrates and later Galen are believed to have practiced techniques of physical therapy as early as 460 BCE, advocating massage, manual therapy techniques, and hydrotherapy to treat patients.

Neurological physical therapy focuses on working with individuals who have a neurological disorder or disease, including but not limited to stroke, chronic back pain, brain injury, and spinal cord injury. Common impairments associated with neurologic conditions include impairments of vision, balance, ambulation, activities of daily living, movement, muscle strength, and loss of functional independence. The techniques involved in neurological physical therapy are wide-ranging and often require specialized training.

Numerous combat veterans have sustained a traumatic brain injury (TBI) as a result of tours of duty in war zones, such as Afghanistan or Iraq. A TBI is caused by an external force, such as an explosive blast. The CDC defines a traumatic brain injury as a disruption in the normal function of the brain that can be caused by a bump, blow, or jolt to the head, or penetrating head injury. Everyone is at risk for a TBI, especially children and older adults. A TBI can manifest as cognitive, physical, behavioral, and/or emotional impairments, and can be temporary or permanent. While TBIs are a serious issue with returning combat veterans, they are not limited to combat situations. The CDC estimates that approximately 1.7 million people each year in the US incur a brain injury as a result of automobile accidents, high contact sports such as football, falling while walking sustaining a head injury, and other injuries to the head.

Stroke occurs when poor blood flow to the brain results in cell death. In 2015 there were about 42.4 million people globally who had previously had a stroke and were still alive. In a stroke, an entire side of the patient can become weakened or paralyzed, known as hemiparesis. It is important to start rehabilitation as soon as possible after a stroke.

In many cases of a severe spinal or brain injury, the path to rehabilitation may be very long or lifelong. In some cases, the progress can be as small as moving a hand or arm 1 cm or being able to have the control and fine motor skills to lift your arm or hand off of the table. When a person has an injury that affects mobility, their own hand becomes dead weight. This is compounded when the brain and nerves are not connecting all the muscles in the arm, and other muscles in the arm may need to take over the job of a paralyzed muscle.

Rehabilitation for TBI and/or stroke patients often involves working on the ability to produce strong movements or the ability to perform tasks using normal patterns. Emphasis is often concentrated on functional tasks and the patient's goals. One technique used to promote motor learning is constraint-induced movement therapy (CIMT). Through continuous practice, the patient relearns to use and adapt an impaired limb during functional activities to create lasting permanent changes. Active participation in CIMT is critical to the motor learning and the recovery process. The focus of CIMT is to combine restraint of the unaffected limb and intensive use of the affected limb. Types of restraints include, for example, a sling or triangular bandage, a splint, a mitt, etc. Due to its high duration of treatment, the therapy has been found to frequently be infeasible when attempts have been made to apply it to clinical situations, and both patients and treating clinicians have reported poor compliance. In the United States, the high duration of the therapy has also made it difficult to get reimbursed in most clinical environments.

CIMT and other repeated movement therapies work because a patient's brain is able to adapt due to neuroplasticity, the ability of the brain to adapt and change throughout an individual's life. Activity-dependent plasticity has been noted as having significant implications for healthy development, learning, memory, and recovery from brain damage.

As stated above, one problem with CIMT is patient compliance. A system that improves patient interest and enjoyment with the therapy would also improve compliance. Another problem stated above is the high cost associated with the specialized training for the medical professional and the time required for therapy.

Another group of people who benefit from physical and/or occupational therapy are those with Autism spectrum disorder (ASD). ASD includes people who are high functioning and can focus obsessively on one side and also includes those who are low functioning such that they have trouble holding a job, performing simple tasks, or even communicating with others. ASD is estimated to affect about 1% of people (62.2 million globally as of 2015), and males are affected more often than females.

Many people with ASD may have additional difficulty with being over stimulated by their environment. There have been many tools used to aid people on the spectrum to learn skills like focus, time management, fine motor skills, language skills, etc. These tools may require significant resources and are thus not available as much as may be needed.

A common comorbidity with ASD is developmental coordination disorder (DCD), also known as developmental dyspraxia or simply dyspraxia. It is a chronic neurological disorder beginning in childhood and is known to affect planning of movements and co-ordination as a result of brain messages not being accurately transmitted to the body. Impairments in skilled motor movements per a child's chronological age interfere with activities of daily living. Various areas of development can be affected by developmental coordination disorder and these will persist into adulthood, as DCD has no known cure.

In addition to the physical impairments, developmental coordination disorder is associated with problems with memory, especially working memory. This typically results in difficulty remembering instructions, difficulty organizing one's time and remembering deadlines, increased propensity to lose things or problems carrying out tasks that require remembering several steps in sequence (such as cooking). Whilst most of the general population experience these problems to some extent, they have a much more significant impact on the lives of dyspraxic people. However, many dyspraxics have excellent long-term memories, despite poor short-term memory. Many dyspraxics benefit from working in a structured environment, as repeating the same routine minimizes difficulty with time-management and allows them to commit procedures to long-term memory.

In addition to Autism spectrum disorder, people who have developmental coordination disorder may also have one or more of these comorbid conditions: Attention deficit hyperactivity disorder (ADHD) (inattention, hyperactivity, impulsive behavior); Dyscalculia (difficulty with mathematics); Dysgraphia (an inability to write neatly or draw); Dyslexia (difficulty with reading and spelling); Hypotonia (low muscle tone); Sensory processing disorder; Specific language impairment (SLI); and, Visual perception deficits.

However, dyspraxics are unlikely to have all of these conditions. The pattern of difficulty varies widely from person to person, and it is important to understand that an area of major weakness for one dyspraxic can be an area of strength or gift for another. For example, while some dyspraxics have difficulty with reading and spelling due to an overlap with dyslexia, or numeracy due to an overlap with dyscalculia, others may have brilliant reading and spelling or mathematical abilities. Some estimates show that up to 50% of dyspraxics have ADHD.

People with developmental coordination disorder sometimes have difficulty moderating the amount of sensory information that their body is constantly sending them, so as a result dyspraxics are prone to sensory overload and panic attacks. Often various coping strategies may be developed, and these can be enhanced through occupational therapy, psychomotor therapy, physiotherapy, speech therapy, or psychological training. As with other conditions listed above, these therapies may be resource intensive and more limited than optimally beneficial to the dyspraxic person.

Yet another problem with the current systems for helping patients in therapy is a lack of a completely objective and reliable method to measure improvement from day to day. There may not be a clear unbiased method to show progress on an incremental level. Individual doctors, nurses, and therapists will all have different biases and approaches and this results in inter-person measurement differences. Even when the same individual takes measurements at different time points and/or different conditions, there will be intra-person measurement differences. The present invention may provide a clear unit of measurement without inter-person or intra-person differences and thus more accurately measure gradual improvement or gradual decline in rehabilitation.

The present invention solves many of the above problems and provides additional utilities in day-to-day life.

### SUMMARY

In one embodiment, the present invention improves neuroplasticity and recovery for patients in need thereof by providing a way to keep patients engaged in repeated practice of using fine motor skills. The present invention is an aid to helping patients through the emotionally and physically demanding journey that may involve creating new neural pathways within the brain and new neural connections to muscles that may have not been used in the same way before. Specifically, the present invention will show a previously drawn object and then step the patient through recreating the object line by line. The system of the present invention can track the patient's recreation and objectively compare each recreation to the original and to other recreations. Objects may be simple drawings, letters of an alphabet, or more complex paintings. The present invention has a system for recording movements as small as 1 or 2 millimeters, recording range of motion as small as 1 or 2 degrees of difference, and pressure sensitivity detection to the capabilities of the stylus used. Thus, the patient may get feedback of improvements in their mobility even if it is difficult or impossible for the naked eye to detect the change. The precise measure of feedback may be a big motivator for a person who needs the small wins as they undergo so much adversity in rehabilitation. Additionally, by tracking and saving each specific recreation, a patient will have more ownership of both the recreation and the therapy process.

In another embodiment of the present invention, a patient can improve speech recovery by recreating an audio performance word for word or with groups of words. As in the first embodiment, the system of the present invention can track the patient's recreation and objectively compare each recreation to the original and to other recreations. Because the system tracks and saves each specific recreation performed by a patient, the patient will have more ownership of both the recreation and the therapy process.

In another embodiment, the present invention improves neuroplasticity and recovery for patients in need thereof by providing a way to keep patients engaged in repeated motion by creating an interesting augmented reality or virtual reality environment for the patient and then requiring the patient to recreate an action or sequence of actions. Such actions may be part of a game, or may be a tutorial. The system of the present invention can track the patient's recreation and objectively compare each recreation to the original and to other recreations.

Another exemplary embodiment of the invention is a system that includes a computer, a touch screen that is coupled to the computer, and a stylus that is coupled to the computer and configured for use with the touch screen. Stored within the computer is a tutorial that includes tutorial data points that when displayed on the touch screen represent an object that is subdividable into portions that are to be redrawn by the patient. The tutorial includes a sequence of steps that are to be performed by the patient. The computer is configured to parse the tutorial data points into a plurality of sets of tutorial data points. Each of the plurality of sets of tutorial data points corresponds to one of the portions of the object that is to be redrawn by the patient using the stylus in combination with the touch screen during one of the sequence of steps of the tutorial. Each of the plurality of sets of tutorial data points has associated with it at least one parameter. The touch screen is configured to display for viewing by the patient a first portion of the object, the first portion of the object has associated with it a first set of tutorial data points. After the first portion of the object is displayed on the touch screen, the computer prompts the patient to attempt to redraw using the stylus in combination with the touch screen the first portion of the object. The computer automatically records patient data points collected from the patient's attempt to redraw the first portion of the object using the stylus in combination with the touch screen. The computer automatically compares the patient data points to the first set of tutorial data points, and when the computer determines that the patient data points are within the at least one parameter for the first set of tutorial data points, the computer automatically prompts the display on the touch screen of a second portion of the object for the patient to redraw, otherwise, if the patient data points are not within the at least one parameter for the first set of tutorial data points, the computer prompts the patient again to attempt to redraw the first portion of the object using the stylus in combination with the touch screen.

In other, more detailed features of the invention, the sequence of steps of the tutorial includes a start step and a finish step, the computer automatically records the patient data points for each of the portions of the object from the start step of the tutorial until the finish step of the tutorial, and the computer prompts the display on the touch screen the patient data points for each of the portions of the object.

In other, more detailed features of the invention, the patient data points include data selected from the group consisting of a pressure of the stylus, an angle of the stylus, and a time associated with use of the stylus. Also, at least one of the portions of the object can include at least one line. In addition, the at least one parameter can be adjustable. Furthermore, the step of the computer automatically recording the patient data points that are collected from the patient's attempt to redraw each of the portions of the object can be repeated for each step of the tutorial until the tutorial is complete.

An exemplary method according to the invention is a method for rehabilitating a patient. The method includes providing a computer; providing a touch screen that is coupled to the computer; providing a stylus that is coupled to the computer and configured for use with the touch screen; and providing a tutorial that is stored within the computer. The tutorial includes tutorial data points that when displayed on the touch screen represent an object that is subdividable into portions that are to be redrawn by the patient. The tutorial includes a sequence of steps that are to be performed by the patient, and the computer is configured to parse the tutorial data points into a plurality of sets of tutorial data points with each of the plurality of sets of data points corresponding to one of the portions of the object that is to be redrawn by the patient using the stylus in combination with the touch screen during one of the sequence of steps of the tutorial. Each of the plurality of sets of tutorial data has associated with it at least one parameter. The method also includes displaying for viewing by the patient a first portion of the object on the touch screen, the first portion of the object has associated with it a first set of tutorial data points; prompting the patient to attempt to redraw using the stylus in combination with the touch screen the first portion of the object that is displayed on the touch screen; automatically recording in the computer patient data points collected from the patient's attempt to redraw the first portion of the object using the stylus in combination with the touch screen; and using the computer to automatically compare the patient data points to the first set of tutorial data points, and when the computer determines that the patient data points are within the at least one parameter for the first set of tutorial data points, using the computer to automatically prompt the display on the touch screen of a second portion of the object for the patient to redraw. Otherwise, if the patient data points are not within the at least one parameter for the first set of tutorial data points, the method includes using the computer to prompt the patient again to attempt to redraw the first portion of the object using the stylus in combination with the touch screen.

In other, more detailed features of the invention, the sequence of steps of the tutorial includes a start step and a finish step, and the method further includes using the computer to automatically record the patient data points for each of the portions of the object from the start step of the tutorial until the finish step of the tutorial; and using the computer to prompt the display on the touch screen the patient data points for each of the portions of the object.

Another exemplary embodiment of the invention is a system for rehabilitating a patient. The system includes a computer, a touch screen that is coupled to the computer, and a stylus that is coupled to the computer and configured for use with the touch screen. Stored within the computer is a tutorial that is configured to prompt the patient to redraw an object that is displayed on the touch screen using the stylus in combination with the touch screen. The object is subdivided into a plurality of portions. The tutorial includes a sequence of steps that are to be performed by the patient. In each of the steps of the sequence of steps, the patient is prompted to redraw at least one portion of the object. Each of the plurality of portions of the image has associated with it at least one parameter. The touch screen is configured to display for viewing by the patient a first portion of the object. After the first portion of the object is displayed on the touch screen, the computer prompts the patient to attempt to redraw using the stylus in combination with the touch screen the first portion of the object. The computer automatically records data collected from the patient's attempt to redraw the first portion of the object using the stylus in combination with the touch screen. The computer automatically compares the data to the first portion of the object, and when the computer determines that the data are within the at least one parameter for the first portion of the object, the computer automatically prompts the display on the touch screen of a second portion of the object for the patient to redraw. Otherwise, if the data are not within the at least one parameter for the first portion of the object, the computer prompts the patient again to attempt to redraw the first portion of the object using the stylus in combination with the touch screen.

In other, more detailed features of the invention, the sequence of steps of the tutorial includes a start step and a finish step; the computer automatically records the data from the patient's attempts to redraw each of the portions of the object from the start step of the tutorial until the finish step of the tutorial; and the computer prompts the display on the touch screen the data for each of the portions of the object.

### BRIEF DESCRIPTION OF THE DRAWINGS

FIG. 1 is a sample view of the present invention and its tutorial and playback feature in one embodiment.

FIG. 2 is a sample view of the present invention and its tutorial feature in one embodiment.

FIG. 3 is one embodiment of a sample view of a menu of featured tutorials and recently added tutorials.

FIG. 4 is one embodiment of a first step of an example tutorial selected from the menu of FIG. 3.

FIG. 5 is one embodiment of several steps after FIG. 4 in the example tutorial.

FIG. 6 is one embodiment of a tutorial creation screen before starting a new tutorial.

FIGS. 7A-C illustrate one embodiment of the present invention for completing several steps for creating an object in a tutorial for writing letters.

FIGS. 8A-C illustrate one embodiment of the present invention for recreating an object in a tutorial for drawing a caricature.

FIGS. 9A-C illustrate one embodiment of the present invention for recreating an object in a tutorial for drawing a caricature.

FIG. 10 illustrates one embodiment of the present invention for recreating an object in a tutorial for drawing a historical picture.

FIG. 11 illustrates various data that may be captured by the system while a teacher or student are using a stylus.

FIG. 12 is a flowchart for one embodiment of a teacher creating a tutorial for drawing an object.

FIG. 13 is a flowchart for one embodiment of a student following a tutorial for drawing an object.

FIG. 14 is a flowchart for one embodiment of a teacher adding audio commentary to a tutorial.

FIGS. 15A-B are a flowchart for one embodiment of a student following a tutorial with audio commentary for drawing an object.

FIGS. 16A-B are a flowchart for one embodiment of a patient improving fine motor skills in occupational, physical, or related therapy.

FIGS. 17A-B are a flowchart for one embodiment of a teacher creating a tutorial for drawing an object while also live streaming the tutorial creation.

### DETAILED DESCRIPTION OF THE PREFERRED EMBODIMENTS

The following definitions are used herein:

Augmented reality (AR): A direct or indirect live view of a physical, real-world environment whose elements are “augmented” by computer-generated perceptual information, ideally across multiple sensory modalities, including visual, auditory, haptic, somatosensory, etc. The overlaid sensory information may be constructive (i.e., additive to the natural environment) or destructive (i.e., masking of the natural environment) and is spatially registered with the physical world such that it is perceived as an immersive aspect of the real environment.

Audio Performance: Any audio recorded using the system of the present invention. An original audio performance is created by a teacher to provide a verbal template to be recreated by one or more students. Audio performance may be speaking, singing, humming, poetry reciting, or other sound recreation.

Cluster: Several lines, words, or actions grouped together by the teacher or the system of the present invention to highlight the style or importance of how the individual line, word, and/or action was originally created to flow with the others in the cluster.

Line: One line is the path travelled by the stylus from the moment it contacts the touch screen and makes one continuous mark until it lifts off the touch screen.

Object: Anything drawn using the system of the present invention. An original object is created by a teacher to provide something to be recreated by one or more students. Objects may be artwork, paintings, cartoons, letters, shapes, kanji, etc.

Physical Performance: Any motion capture recorded using the system of the present invention. Motion capture may be recorded using virtual reality equipment, video equipment, etc. An original action performance is created by a teacher to provide a physical template to be recreated by one or more students. Action performances may be dance, tai chi, yoga, mock sports practice (such as archery, golf, bowling, tennis, baseball, etc.), and other physical motions to be recreated.

Step: An incremental portion of a tutorial that may have been determined automatically by the system or manually by a teacher. A step is one or more data points grouped as a unit to be recreated by a student within a set of parameters. For example, a step may be a single line or cluster of lines in an object; a step may be coloring one or more parts of an object; a step may be a word or a group of words to be repeated by a student in an audio performance; and/or a step may be a single movement or sequence of movements in a physical performance.

Student: A person following the steps of a tutorial to recreate an object, audio performance, and/or physical performance. For physical therapy/occupational therapy, a student is a patient.

Stylus: An object that a student or teacher uses to add content to a touch screen. Examples include, but are not limited to, a finger, an Apple Pencil®, a stylus pen, a Logitech® Crayon, a Microsoft® Surface Pen, etc.

Teacher: A person creating an object to be recreated using the system of the present invention. In some embodiment, more than one teacher may work together to create a tutorial, especially if there is more than one student for the tutorial, such as a physical performance tutorial of a dance with two partners.

Touch Screen: A touch sensitive screen on an electronic device. A teacher or student may add content to the touch screen with a stylus.

Tutorial: A series of steps for a student to follow from start to finish to recreate the action performance, audio performance, and/or object created by a teacher.

Virtual Reality (VR): A computer-generated scenario that simulates a realistic experience in an immersive environment that can be similar to the real world in order to create a lifelike experience grounded in reality, or a science fiction or fantasy environment and/or experience.

The system of the present invention improves neuroplasticity and recovery of patients in need thereof by improving engagement in repeated practice of motions. One theory behind this improved engagement is that the present invention taps into the “IKEA effect” for students. According to Wikipedia, the IKEA effect is a cognitive bias in which consumers place a disproportionately high value on products they partially created. The name derives from the name of Swedish manufacturer and furniture retailer IKEA, which sells many furniture products that require assembly. Michael I. Norton of Harvard Business School, Daniel Mochon of Yale, and Dan Ariely of Duke described the IKEA effect as “labor alone can be sufficient to induce greater liking for the fruits of one's labor: even constructing a standardized bureau, an arduous, solitary task, can lead people to overvalue their (often poorly constructed) creations.”

Initiation rituals for joining groups date back to some of humanity's earliest records, causing members to perceive membership in those groups as having greater value. This is the IKEA effect phenomenon before IKEA, and had been noted by others previously. For example, a 1959 study by Aronson and Mills that has been described as a “classic,” produced results that seem to reflect either the IKEA effect or a closely related phenomenon. Female participants were required to undergo “no initiation, a mild initiation, or severe initiation” before entering a discussion group. The women's later appraisal of the group's value was proportional to the effort that had been demanded of them before being allowed into the group.

Combined with other features, the present invention builds upon the IKEA effect in that the patient/student must use their own effort to follow a tutorial, and the system records the student's actual work instead of an idealized version. Because of this added effort, and an increased sense of owning the result of the tutorial, the student will take increased ownership in completing tutorials and the recovery process.

In one embodiment, the present invention may be used as a learning tool to aid people on the Autism spectrum. The system may especially aid those who are overstimulated in common environments and when they see too many choices or steps in front of them.

The way the present invention aids to combat over stimulation is by only showing the student the next step in the tutorial. The student only needs to focus on one step at a time. The student may feel less anxiety because they are not overwhelmed with the big picture. They also may not be as stressed because the present invention stops after each step so the student may control the time it takes to complete the tutorial. The present invention is also an example of cost savings to the person on the spectrum as well as any support members aiding them.

The present invention may be used to gamify an otherwise difficult or boring therapy, rehabilitation, or lesson to encourage a student to continue with therapy and/or rehabilitation and/or learning.

The present invention may also give a student motivation by way of an accomplished drawing that the student knows they crafted.

The present invention may also help with a person's confidence in personal skills and in life in general. There are many studies that show when a person is learning a new skill, even as they are learning and as they get closer to mastering that skill, different areas of the brain light up. Even places that are not related to the current task at hand. This also increases neural plasticity.

Studies also show how people who are given the opportunity to feel creative before they need to perform a stressful or demanding task perform better in the stressful task. This is amplified further in cases where a person has a learning disability. Because people with learning disabilities may need more time to learn, the present invention is a great example of how a person can learn at their own pace and build confidence and the person can do this on their terms and in private. And yet the results and progress can be shared and tracked without the need for constant one on one monitoring by a teaching professional.

In one embodiment of the present invention, a teacher records the creation of a tutorial using the system. The tutorial may show how to create a drawing, painting, or writing style; performing an audio presentation such as speaking a new language or a theatrical play; or a physical performance such as tai chi, a golf swing, etc. The tutorial may include undos and erases, etc. The present invention takes the recorded tutorial and parses the data into a series of sequential steps. For non-audio tutorials, the system may also record verbal commentary by the teacher to help explain a step, cluster, or other item of note. The tutorial will be turned into sequential steps from start to finish for a student to follow to recreate the object in the tutorial. The tutorial may request the student to repeat a step one or more times if the student did not complete the step within the parameters set by the teacher or the system.

A video preview and/or a GIF preview of parts of the tutorial and/or the entire tutorial may be auto created by the system of the present invention.

In one embodiment of the present invention, a student may use the system to follow, redraw, trace over, or copy an object in a tutorial, for example, with artwork, drawings, etc. The student may add new drawings to the object, erase content from the object, change colors, add or take away special effects, etc. If the teacher provides the relevant permission, the student may share the new art with others or sell it to others.

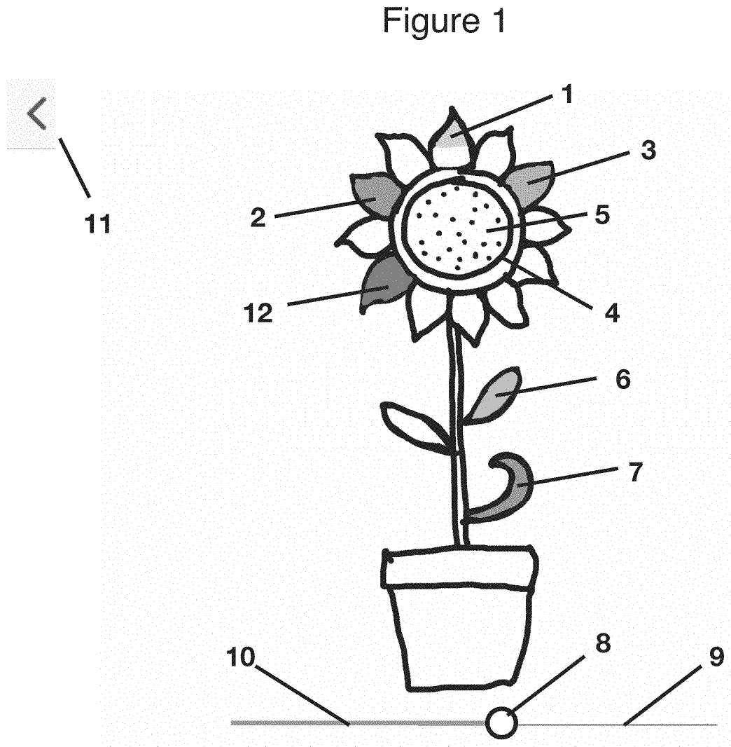

In reference to the Figures, FIG. 1 shows one embodiment of a tutorial for drawing and coloring a flower. Yellow color 1 has been chosen by the teacher for the tip of the petal, and will auto appear during the tutorial as a student draws over the line(s) in the correct step. Gold color 2 and orange color 3 were chosen by a teacher to be added after yellow color 1, and will appear sequentially during the tutorial as a student reaches the relevant step(s). The colors may be added automatically or require student action. Line 4 appears at the relevant step for the student to draw over. Dots 5 appear in the step after line 4. Green color 6 and dark green color 7 will appear sequentially during the tutorial as a student draws over each during the relevant steps. Completion indicator 8 shows how far along a student is in following the tutorial. Grey line 9 indicates the amount of the tutorial remaining and highlighted line 10 indicates the amount of the tutorial completed. Back arrow 11 will return a student to the previous screen. Purple color 12 is a color selected by a student as a variant coloring option not mandated by or selected by the teacher in one embodiment of the tutorial.

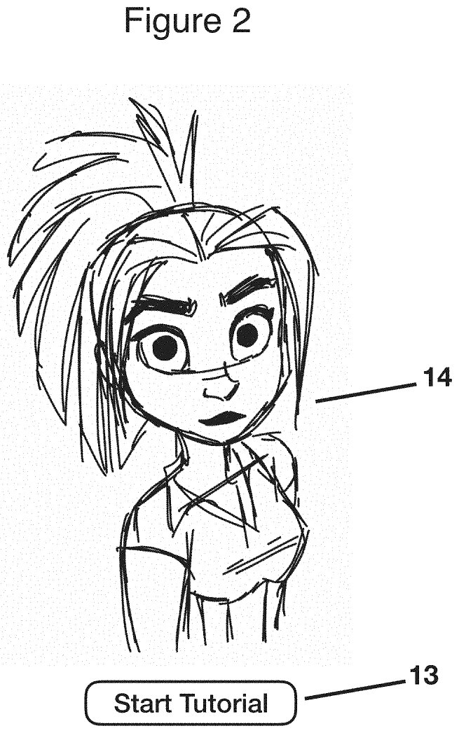

In reference to FIG. 2, start button 13 will start the tutorial of how to draw picture 14. In one embodiment, the system may quickly go through each of the sequential steps needed to go from a blank screen to the final picture after a student selects start button 13. The tutorial will then return to the first step for the student to trace.

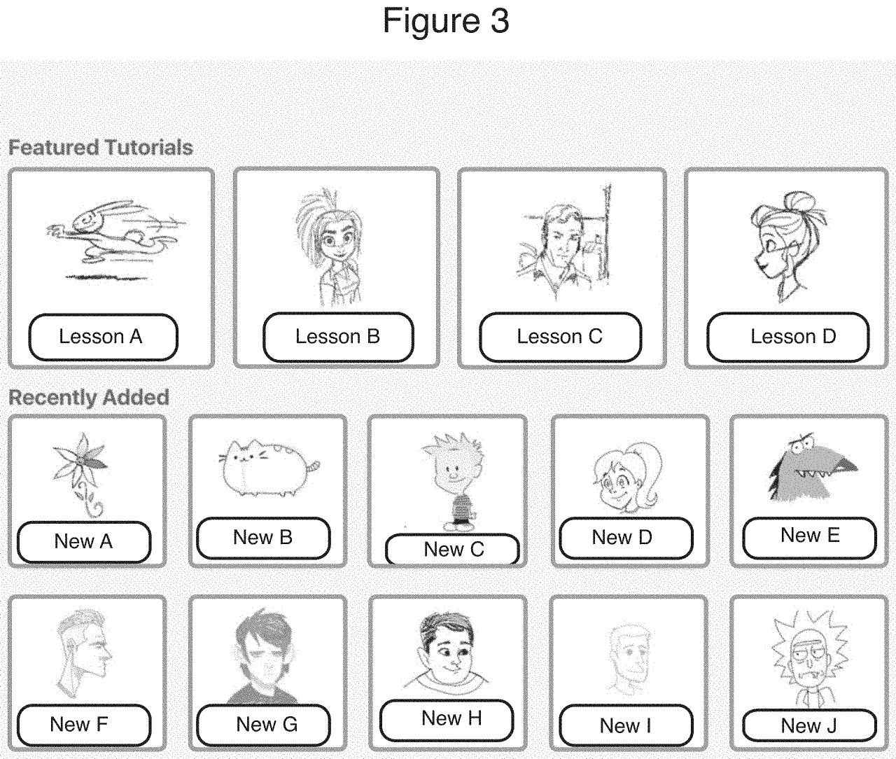

FIG. 3 shows one embodiment of a menu of featured tutorials and recently added tutorials.

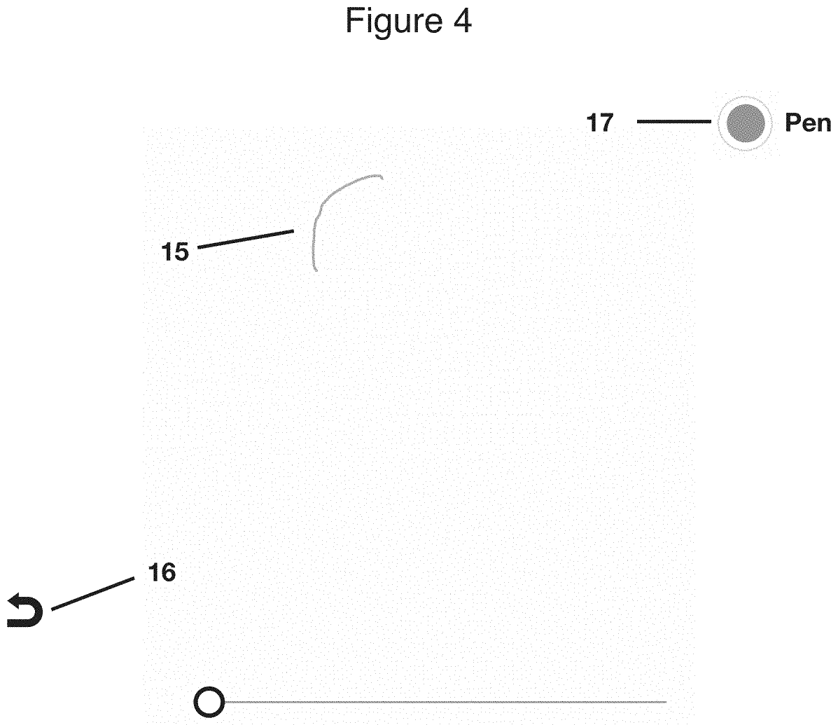

FIG. 4 shows line 15, the first step for tracing the “michael cain” tutorial from the featured tutorials of FIG. 3. Undo button 16 allows a student to go backwards to redraw the previous step. Stylus indicator 17 shows the current pen and color in use for drawing a line.

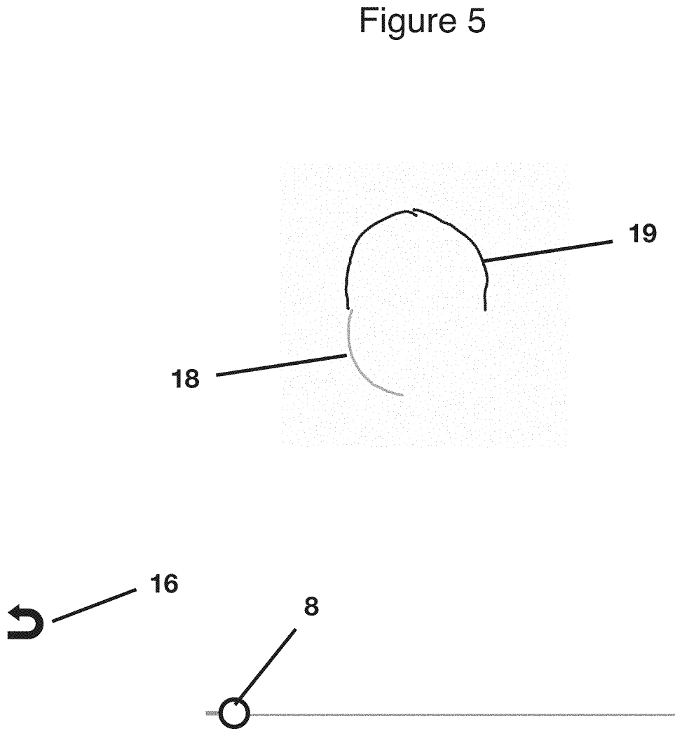

In reference to FIG. 5, line 18 is highlighted to indicate redrawing this line is the next step in the tutorial. Line 19 is the previous line drawn by the student. Completion indicator 8 shows the student's progress.

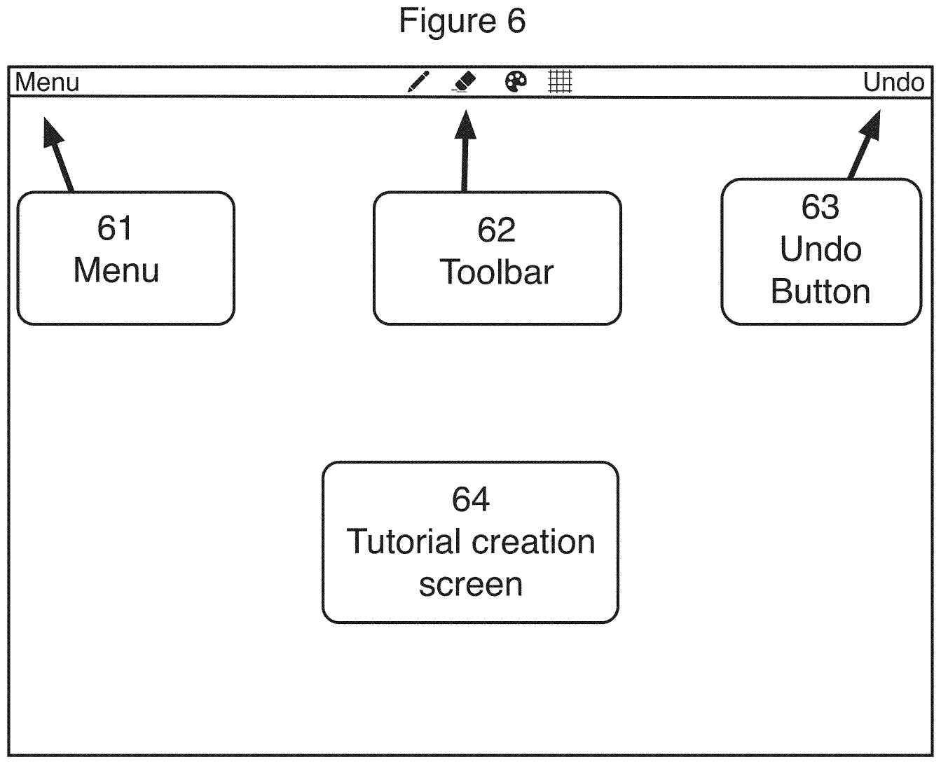

FIG. 6 shows one embodiment of a blank tutorial creation screen before starting the creation of a tutorial. Menu 61 allows a teacher to navigate to a different page. Toolbar 62 allows the teacher to select and change between tools. Examples of tools include but are not limited to: pens; pencils; brushes (including but not limited to brushes that imitate acrylic paint, water colors, and/or oil paint); various calligraphy brushes; erasers; color palettes; selections; opacities; layers; etc. The tools may be adjusted for color, thickness, and/or textures (including, but not limited to: crayon, chalk, colored pencil, graphite pencil, carpenter pencil, technical pencil, pastels, white paper, rice paper, brown paper, cotton paper, chalkboard, slate, eggshell, grid paper, grid dots paper, etc.). Undo button 63 allows a teacher to undo the previous step. Selecting undo button 63 multiple times may allow for multiple steps to be removed. Tutorial screen 64 is blank by default at the beginning of tutorial creation, but may optionally have some elements as background which would not be recreated by a student.

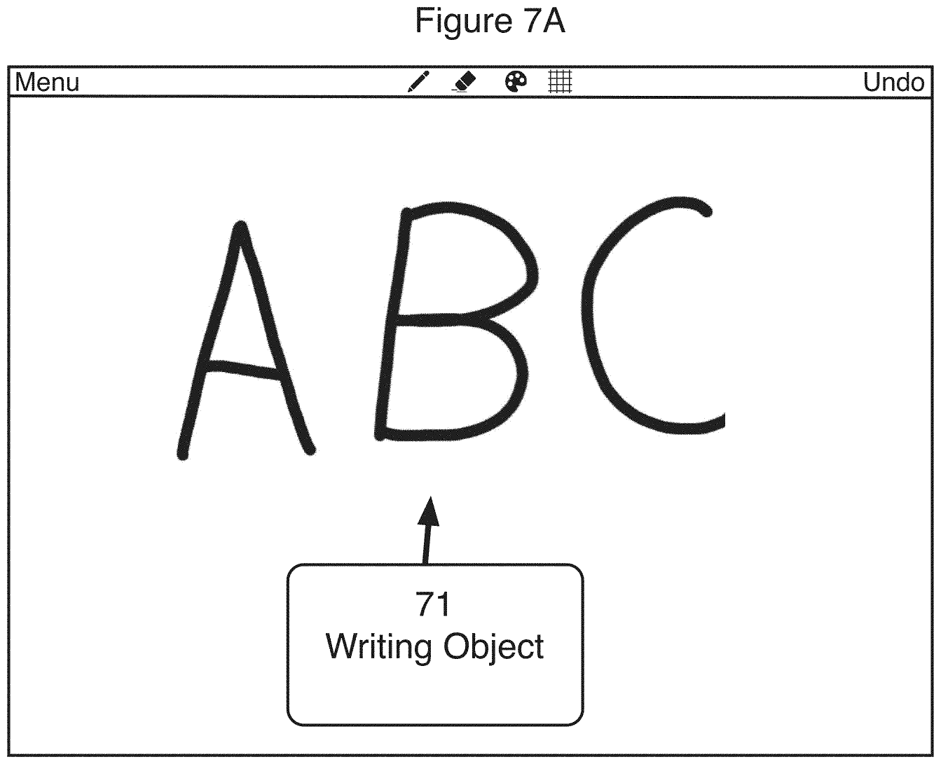

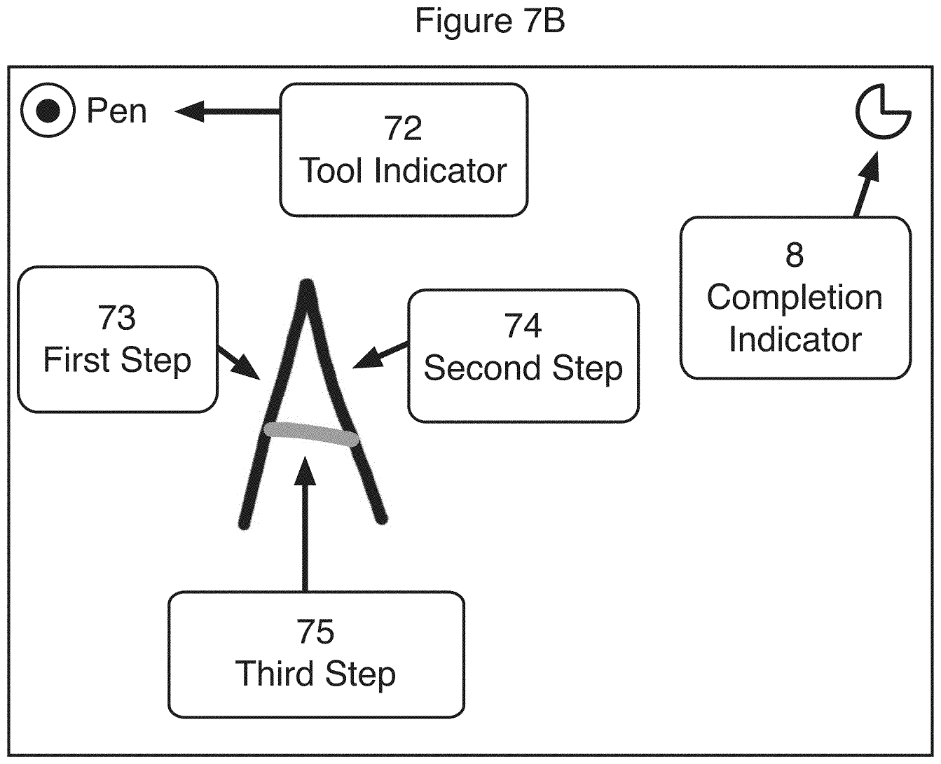

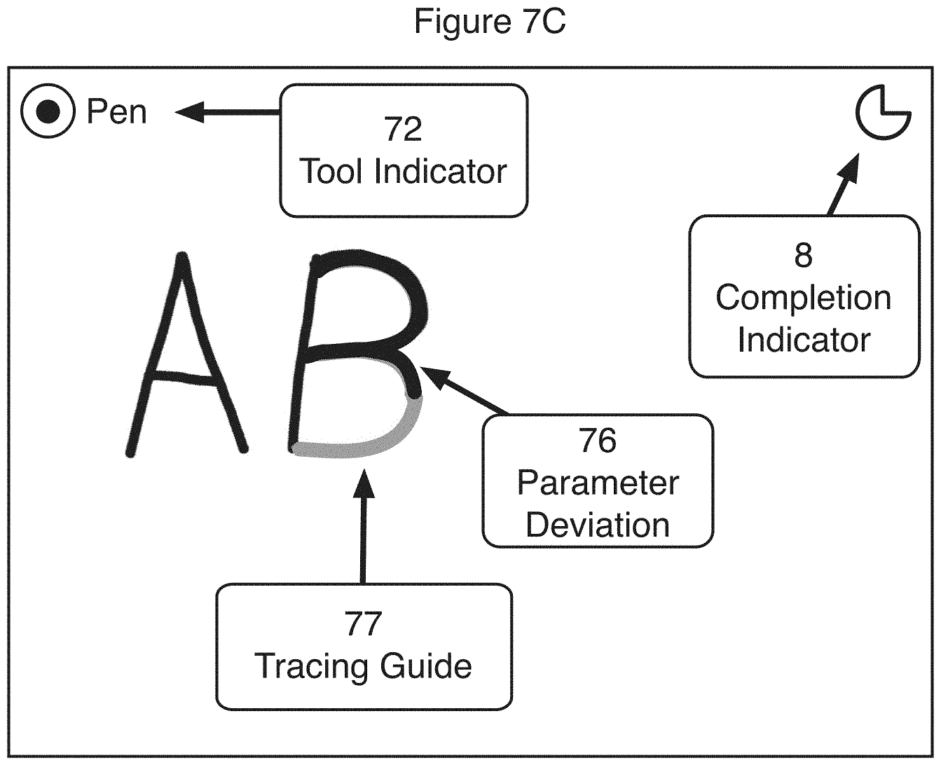

FIG. 7A shows an embodiment of a tutorial for writing letters. Writing object 71 are the letters ABC, created by the teacher in a specific order to be followed by a student.

FIG. 7B shows the third step of the tutorial for recreating writing object 71. Tool indicator 72 shows the currently active tool, and in this example, the size of the lines to be drawn. First step 73 and second step 74 are completed. The system is prompting the student to recreate third step 75 by drawing a line between the midpoints of the line completed in first step 73 and the line completed in second step 74. Completion indicator 8 shows the amount of the tutorial completed relative to the total number of steps. In FIG. 7B, completion indicator 8 is a simple pie chart indicator and shows that the tutorial is about 33% complete.

FIG. 7C illustrates when a student has exceeded a parameter deviation 76 (the tracing is outside the lines more than allowed by the initial parameters set by the teacher). The system stops the recreation of the object and waits for the student to repeat all or part of the current step before proceeding to the next step in the sequence. If the student is unable to meet the parameter deviation requirements, the system may relax the parameters in subsequent attempts to complete the step.

Trace guide 77 is an example of the tracer line/guide line that is a reproduction of the exact drawing by the teacher. Trace guide 77 may be in a different color or pattern from other lines on the page to attract attention to the line and to make clearer that this is the line to recreate for the current step. In FIG. 7C, completion indicator 8 shows the student has completed about 66% of the tutorial.

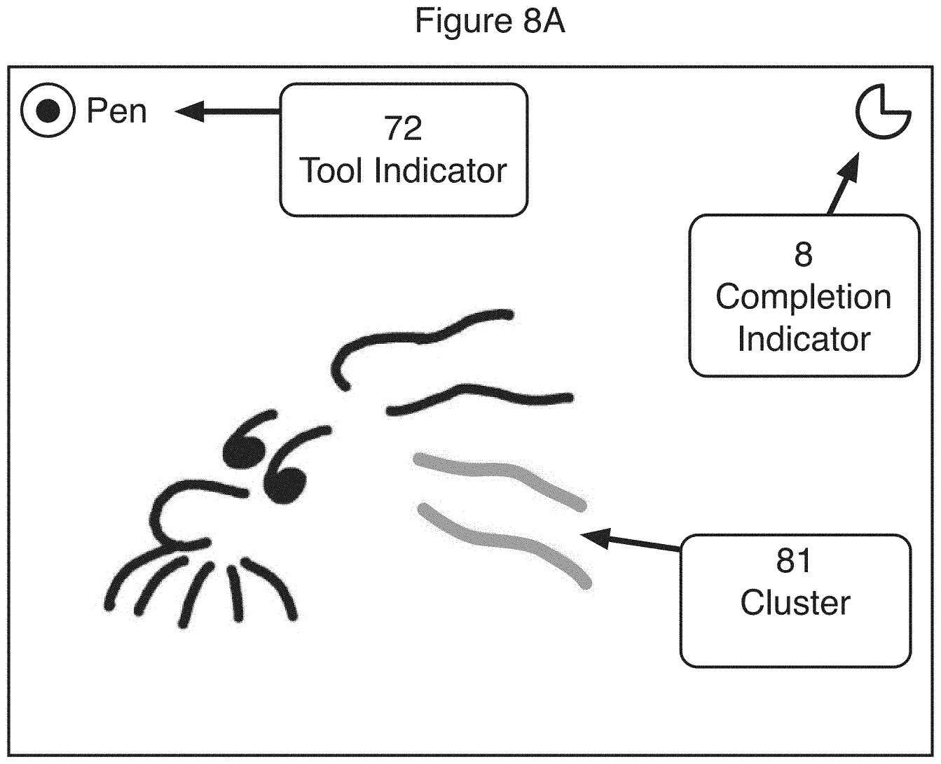

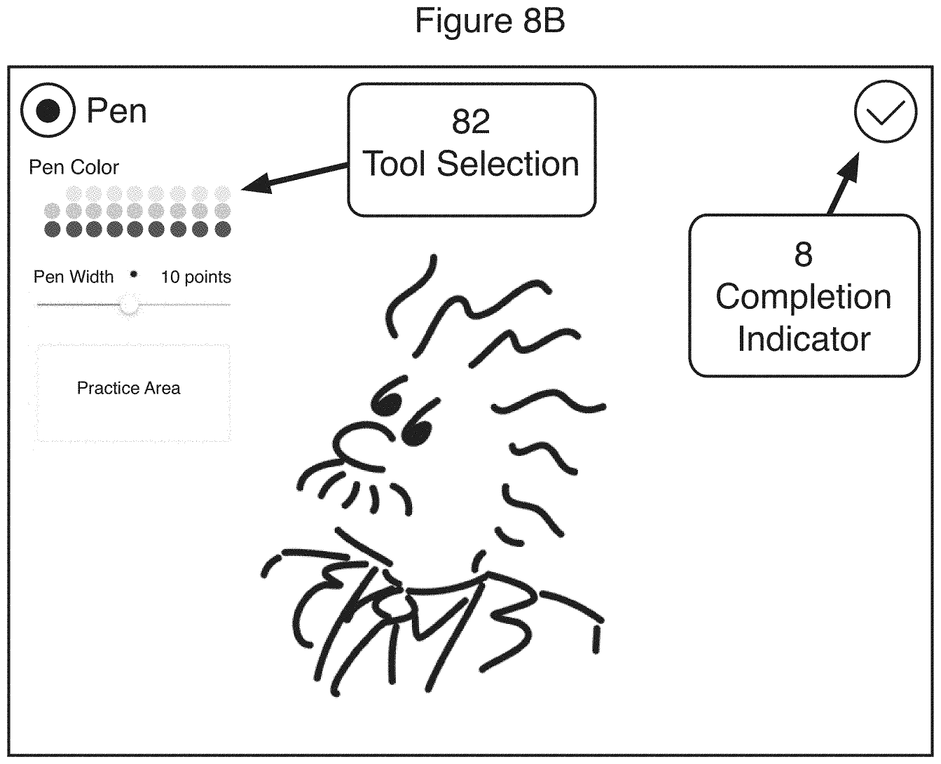

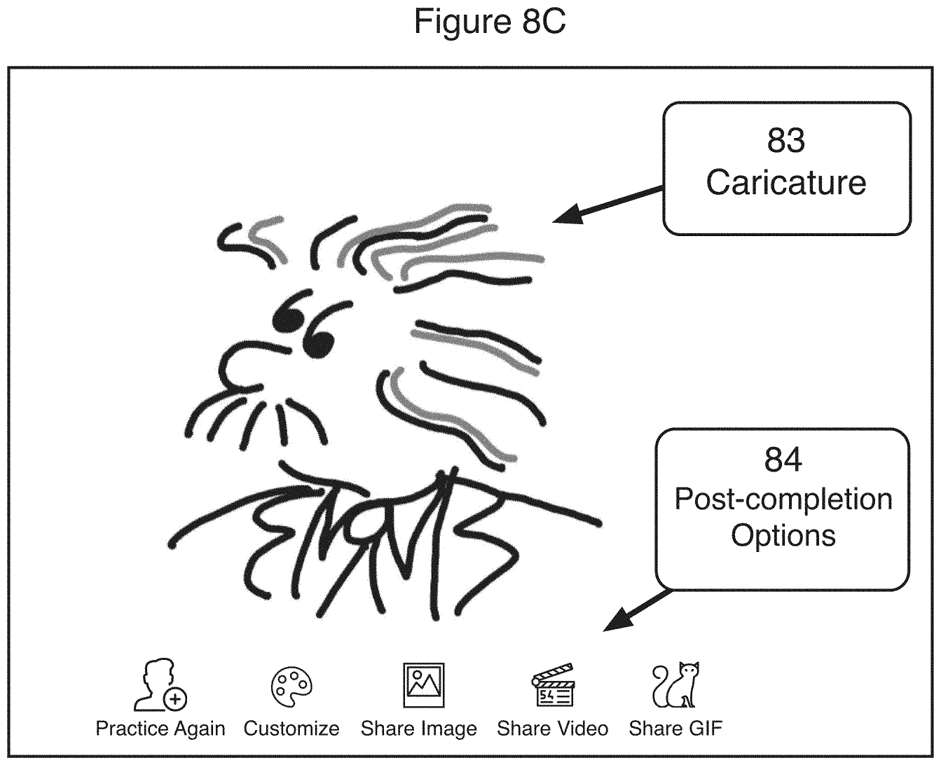

FIG. 8A illustrates an example where two lines were drawn in a sequence by a teacher. The system grouped the two lines in cluster 81 together in one step so that the student recreates both lines before moving to the next step.

FIG. 8B illustrates how customization may take place after a tutorial is completed, as shown by a checked box in completion indicator 8. In FIG. 8B, a student has opened tool selection 82 and may select alternate colors and pen widths to be able to modify the art without guidance, thus making it more their own.

FIG. 8C illustrates how caricature 83 was modified from FIG. 8B with additional lines for hair in a different shade or color. Post-completion options 84 is an example of options after completion of a tutorial. The student may want to practice again, customize the object, and/or share what they have drawn. In one embodiment, the present invention may have pre-completion options. In other words, a student may have an option to add and/or customize the object with their own content prior to completion of a tutorial.

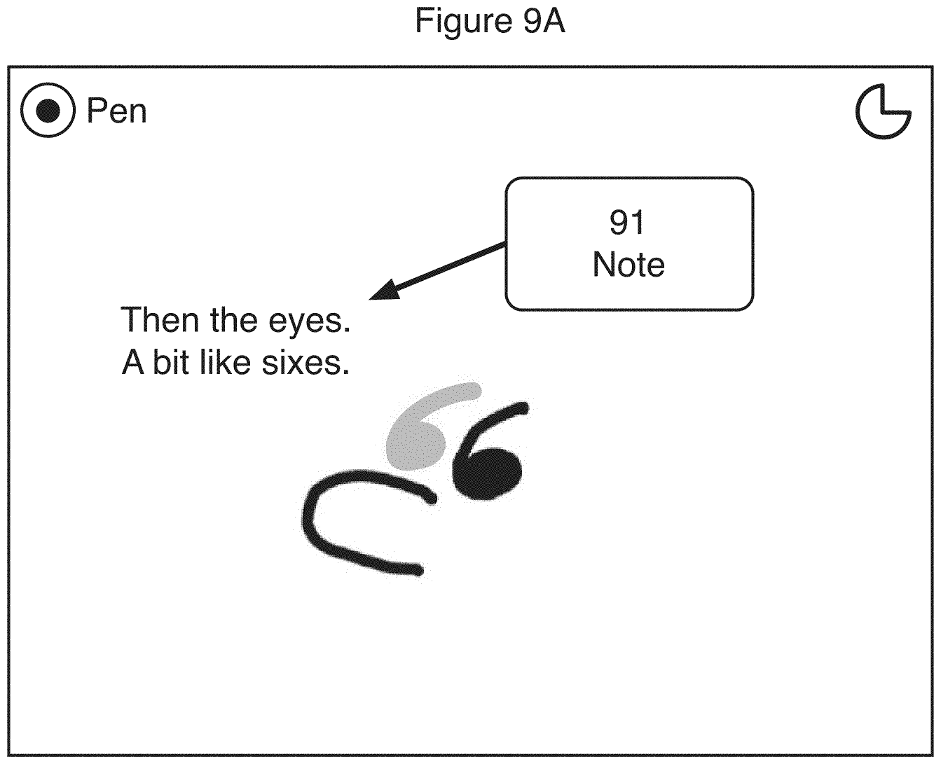

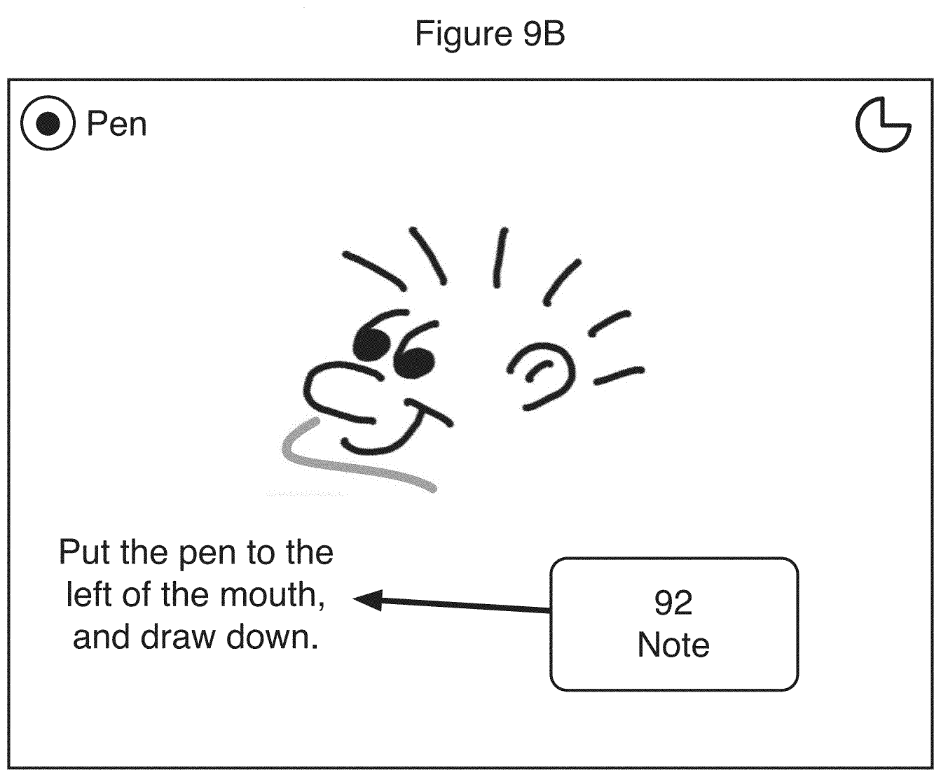

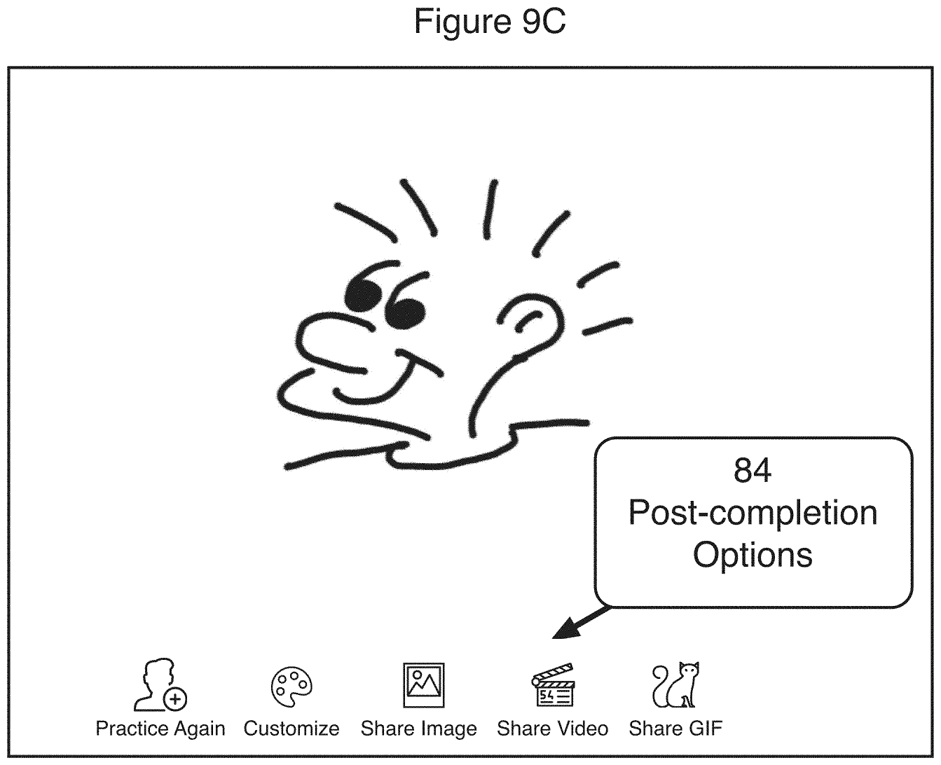

FIG. 9A illustrates an example of a teacher adding customized notes at one or more steps in the tutorial. Note 91 in this example provides a hint for drawing the eyes. Notes may be customized to be at different locations on the page, have an animated effect when the note appears, and/or may be designed to appear only for certain steps or when a student has triggered a parameter deviation.

FIG. 9B illustrates an example of a teacher providing detailed help in completing a step. Note 92 illustrates how a teacher may add additional encouragement and comfort for a student with special learning needs. A student with a learning disability may need reassurance that they are given permission to draw and they may have a different context to remember how to complete the current step for recreating the object. As with FIG. 8C, post-completion options 84 may be available after a student finished a tutorial. The teacher may set up a note to suggest one or more post-completion options 84 after the last tutorial step is finished.

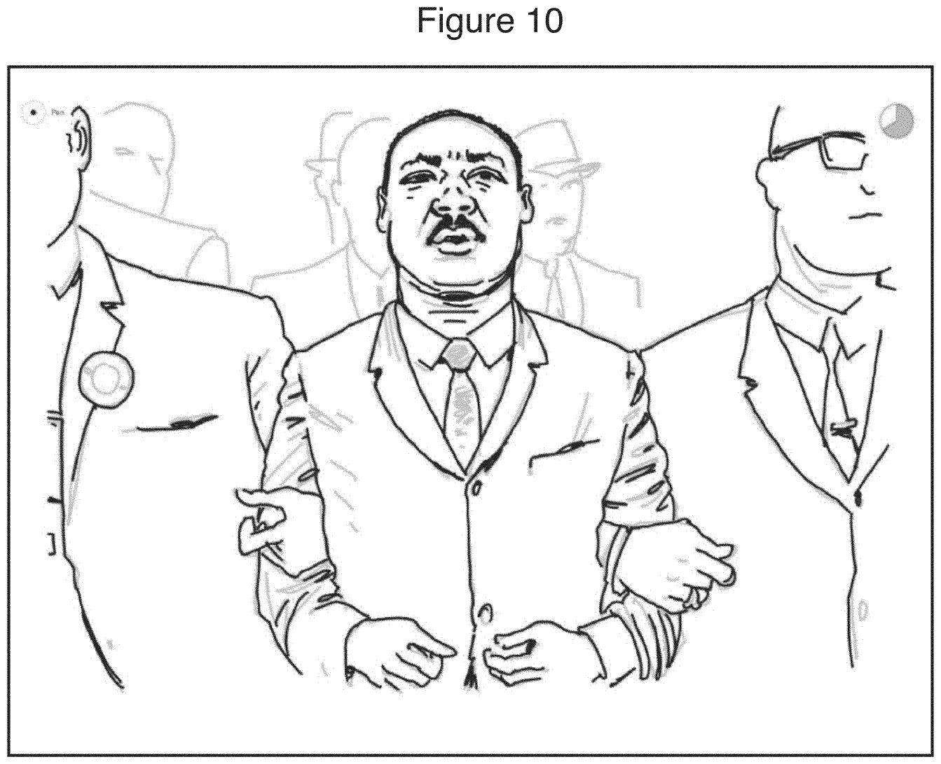

FIG. 10 illustrates a drawing of a historical event or picture. An audio file may play as steps are completed as the student progresses in the drawing. In this example, Dr. Martin Luther King's “I have a dream” speech may start at the beginning of the first step or at later steps. Audio may play other quotes or speeches by him. There may not be an on-screen indicator of audio playing and this is because it may be part of the teaching lesson that the reward of the audio file is variable.

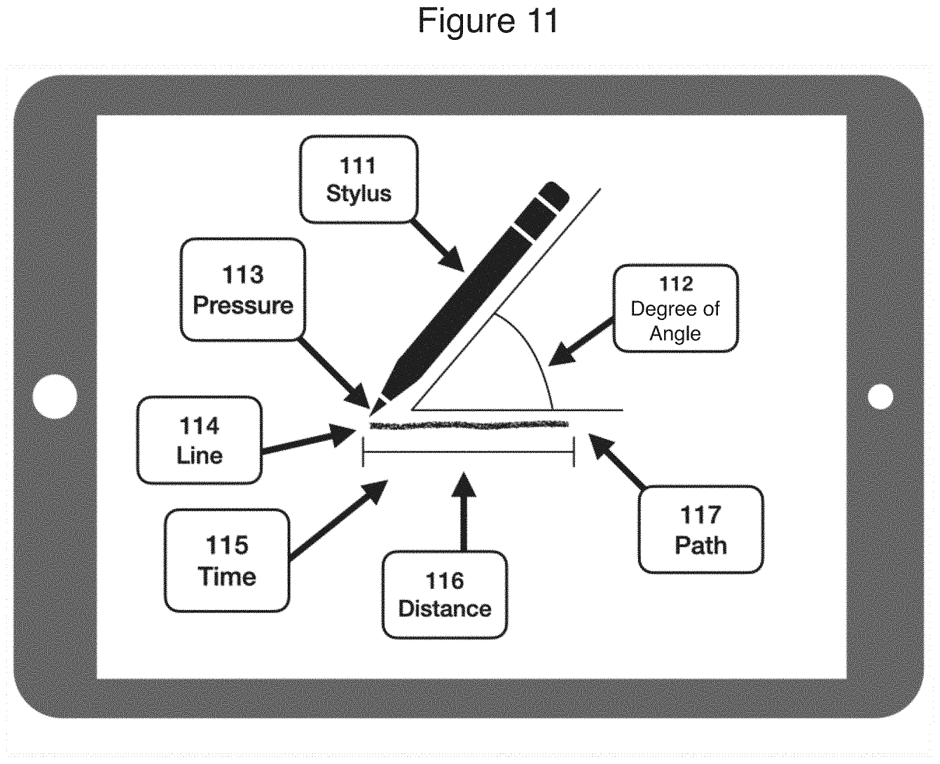

FIG. 11 illustrates various data that may be captured by the system while a teacher or student is recreating an object. In a preferred embodiment, stylus 111 is an Apple Pencil®, a Logitech® Crayon, a Microsoft® Surface Pen, or similar stylus that allows angle degree 112 and/or amount of pressure 113 applied to screen to be recorded while line 114 is drawn. If the screen itself allows for recording pressure sensitivity applied, then a finger or mechanical stylus may be used and the system may still capture pressure 113 data. Data for degree 112 may be captured for some VR or motion capture systems with a finger or mechanical stylus. The system may also capture time 115 for stylus to complete the line from start to finish. The system may also capture the total distance 116 of the time and path 117 of the line.

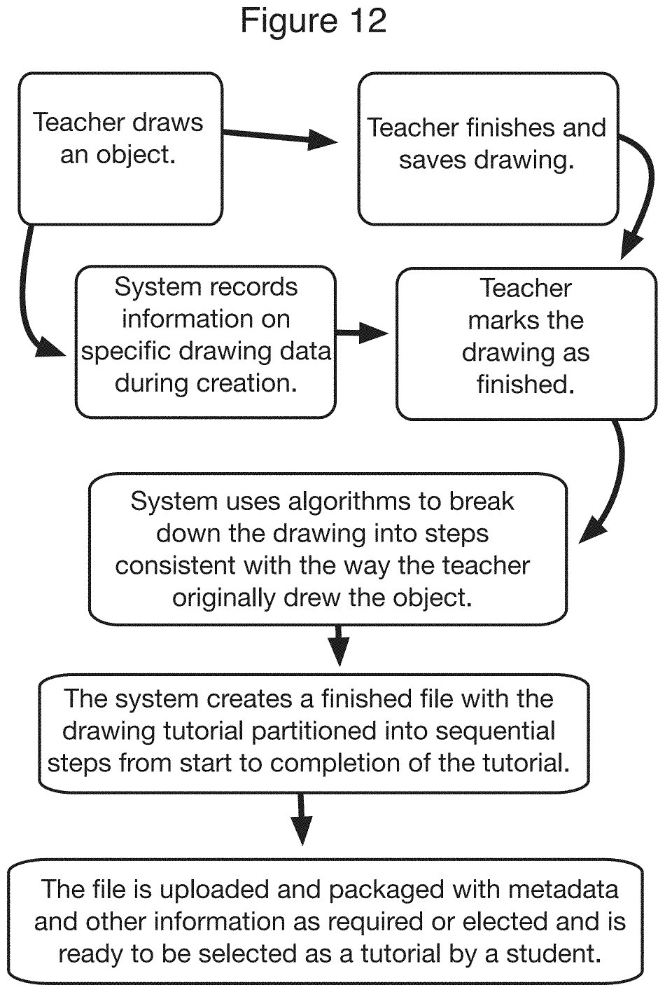

FIG. 12 is a flowchart for one embodiment of a teacher creating a tutorial for drawing an object.

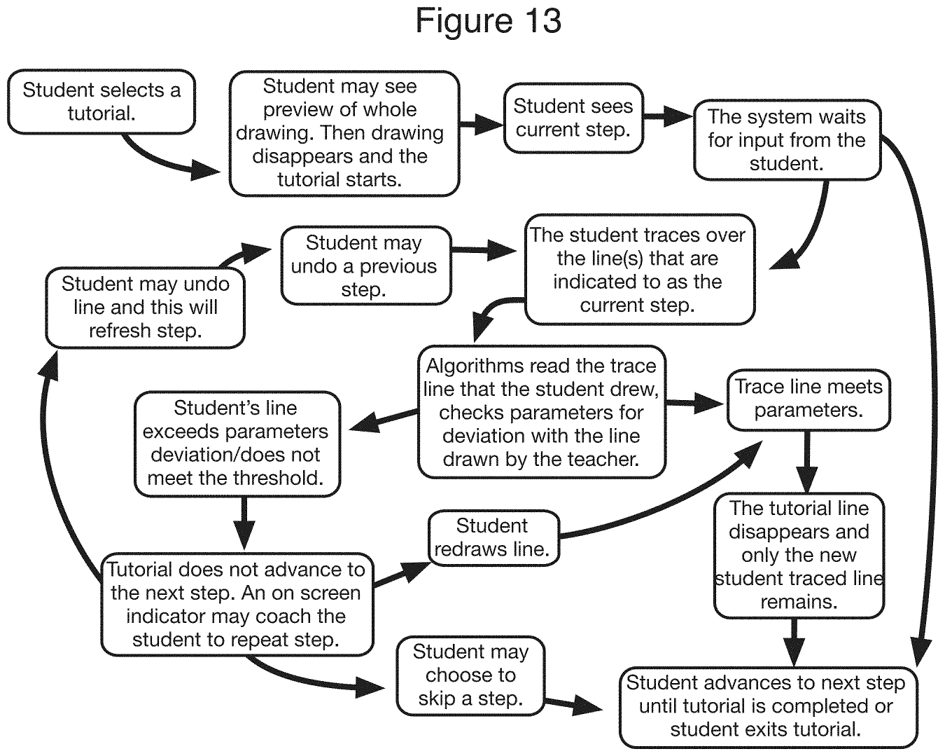

FIG. 13 is a flowchart for one embodiment of a student following a tutorial for drawing an object.

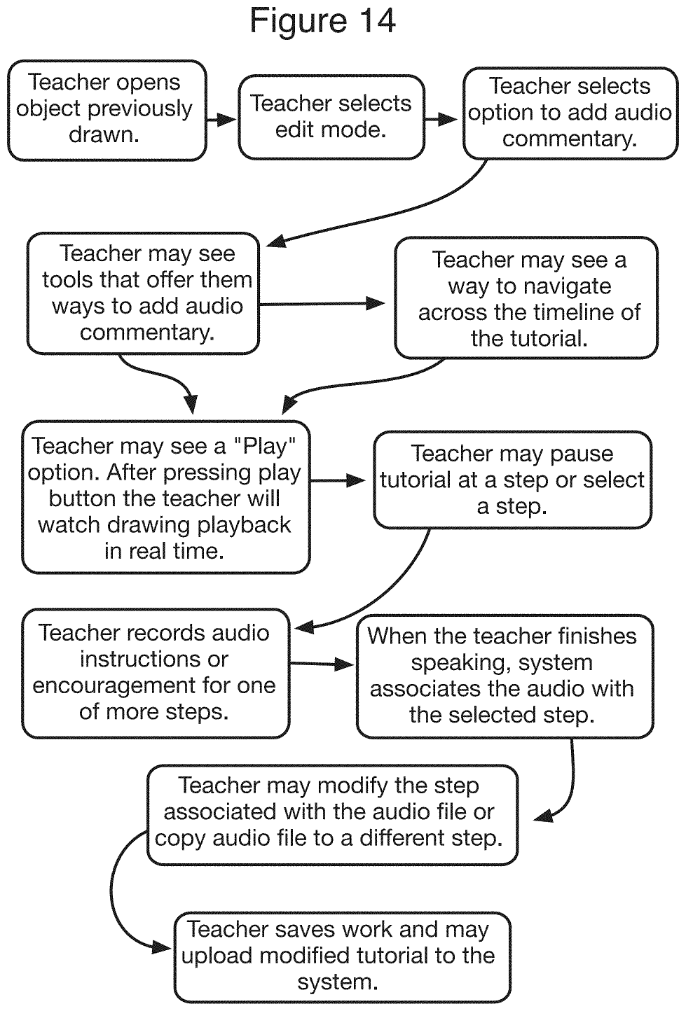

FIG. 14 is a flowchart for one embodiment of a teacher adding audio commentary to a tutorial.

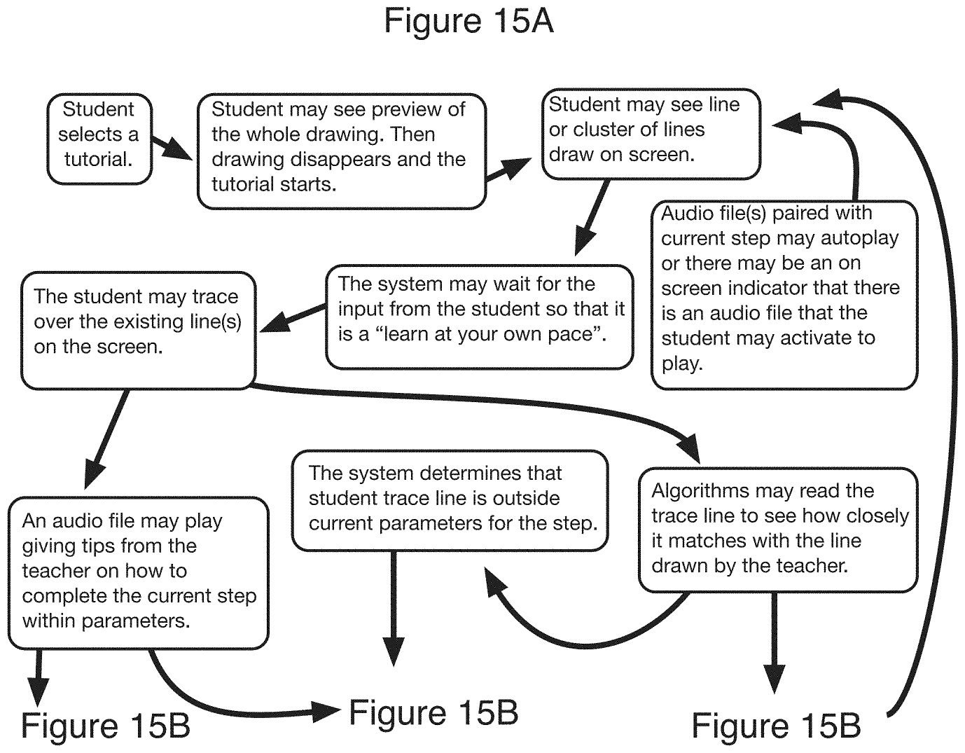

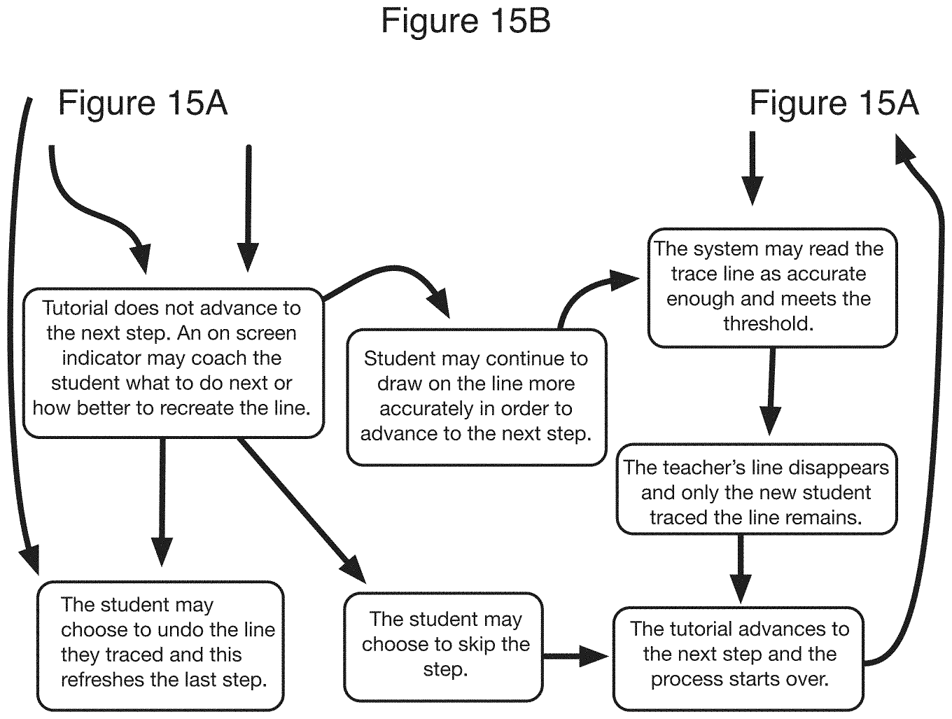

FIGS. 15A-B are a flowchart for one embodiment of a student following a tutorial with audio commentary for drawing an object.

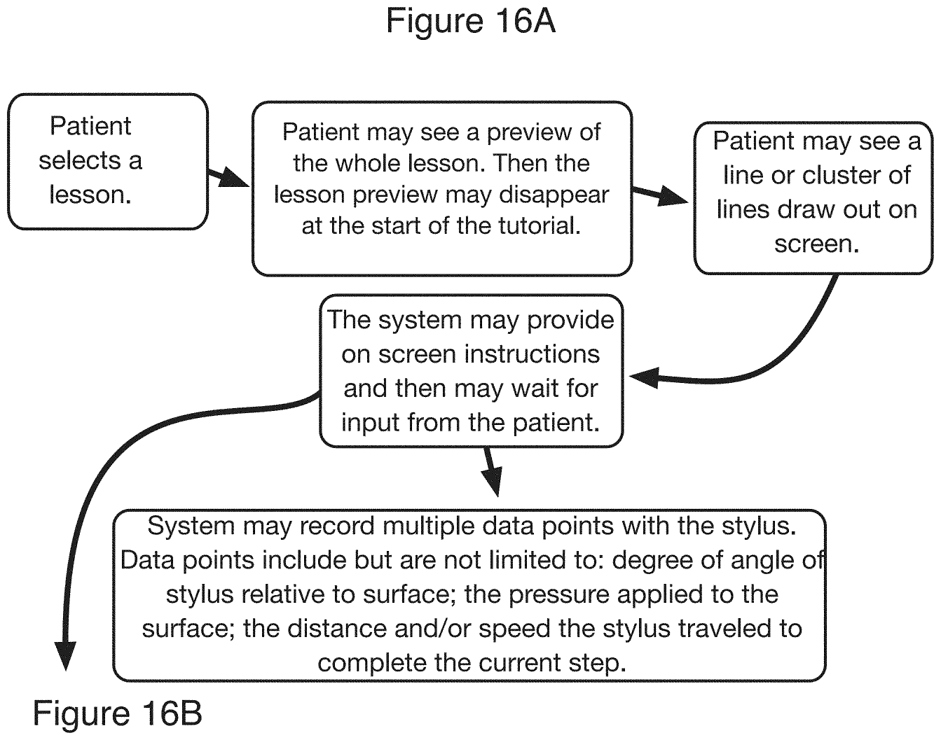

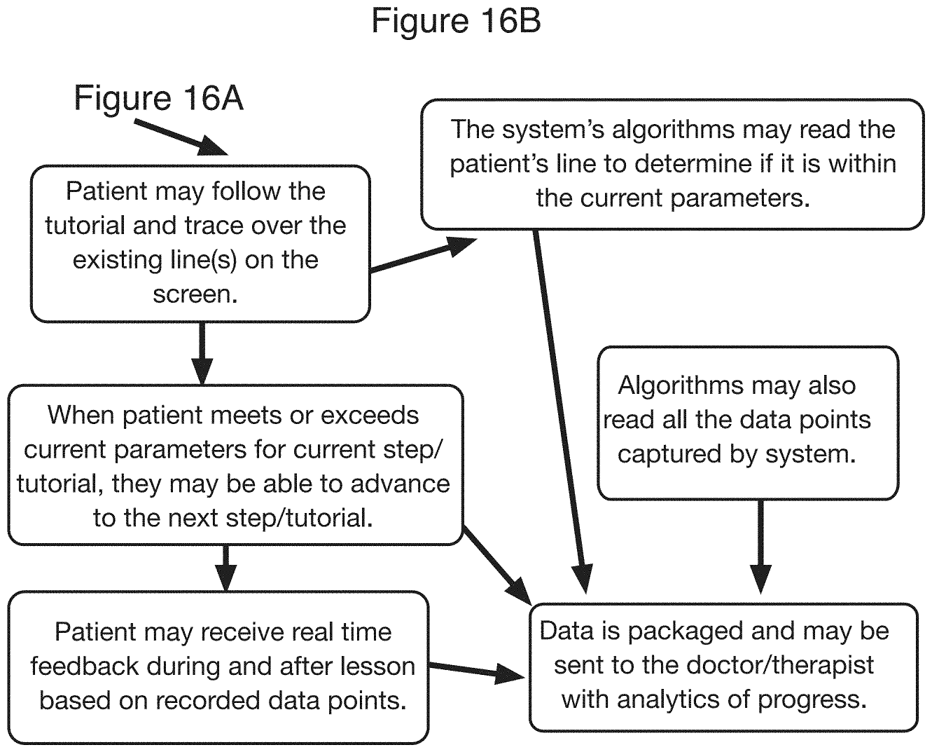

FIGS. 16A-B are a flowchart for one embodiment of a patient improving fine motor skills in occupational, physical, or related therapy.

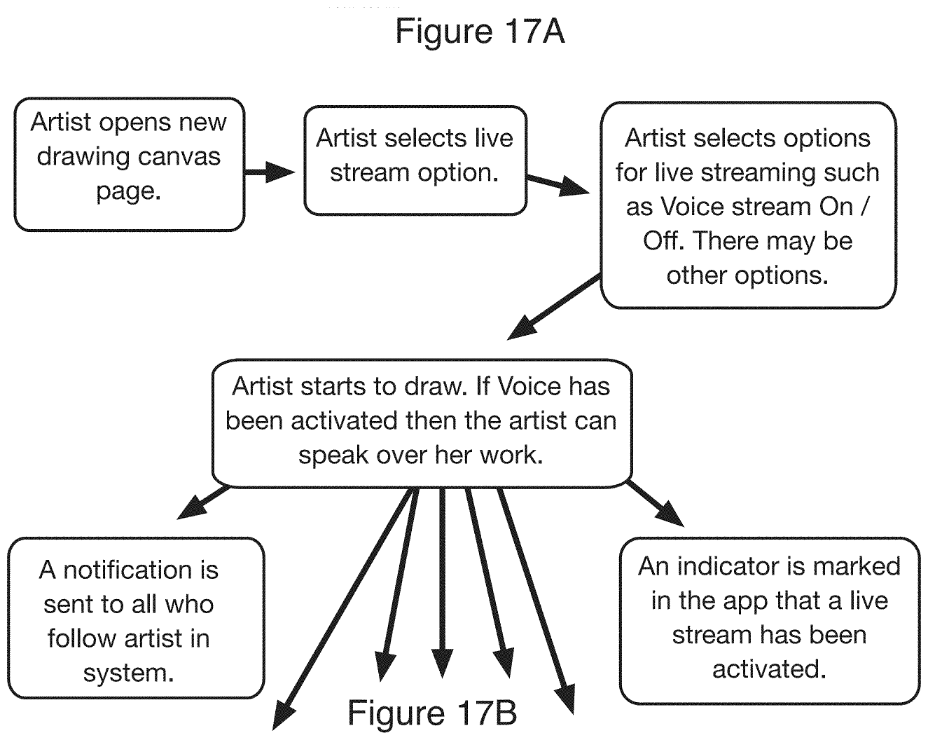

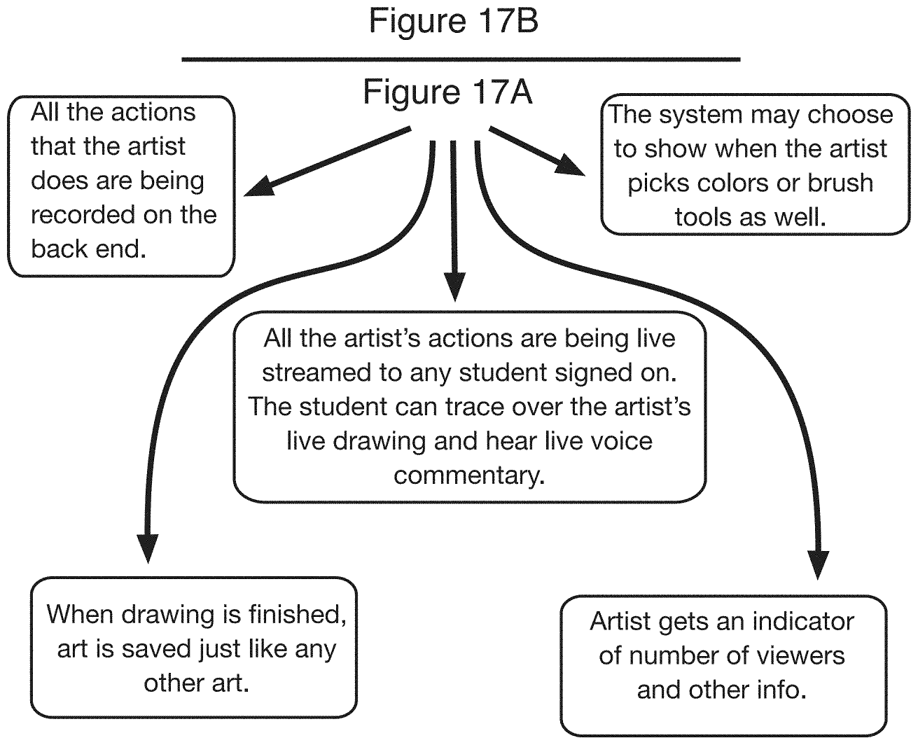

FIGS. 17A-B are a flowchart for one embodiment of a teacher creating a tutorial for drawing an object while also live streaming the tutorial creation.

Illustrations of the drawings and examples are for the purpose of describing selected embodiments of the present invention and are not intended to limit the scope to only these illustrations and examples.

### EXAMPLE 1

### Improving Neuroplasticity and Recovery—Stroke Recovery, Writing Name

In one prophetic example, a stroke patient needs to learn how to write their name with their previously non-dominant hand. The patient writes their name within the system of the present invention at the start of therapy as an initial reference. Medical staff or the patient may also scan in their former signature or an ideal signature as a point of reference. At the end of the first day, the patient may compare the three signatures in order to show improvement. Measurements towards a reference score may include any of the data points recorded by the system.

### EXAMPLE 2

### Improving Neuroplasticity and Recovery—Holding Cup

In one prophetic example, a patient in physical, occupational, and/or related therapy may need to practice the movement of holding a cup or another object. The patient may not have the strength yet to hold a physical cup. The present invention supplies an Augmented reality component and/or a Virtual reality component whereby the patient may use the system to see a virtual cup. The movements of the patient are recorded and analyzed relative to a cup holding tutorial so that the patient may relearn the exact movements. As a variable reward to keep the patient motivated, the VR or AR world may display a sword or lightsaber or any other fantasy object for the patient to grasp or hold.

### EXAMPLE 3

### Physical Performance—Occupational Therapy, AR Holding Ball

In a prophetic example, a patient looks at a screen connected to a camera and the system displays a virtual ball for the patient to hold. The ball may be something that is more motivating to the patient/student like a crystal ball or a planet or the Death Star or a ball of flames. The patient will be guided through a series of movements as directed by a tutorial, doctor, or other medical professional. The patient may be asked to cradle the ball or pass it from one hand to another, or to close their fist and crush the ball.

This tutorial may be made by the teacher doing the same movements and the present invention would record the movements and mark out what paths through the space were made in order to recreate them.

### EXAMPLE 4

### Improving Neuroplasticity and Recovery—Physical Performance—Physical Therapy, Stretching Within VR Game

In a prophetic example, a patient needs to stretch their arms in a way that is painful or uncomfortable. Within the VR space, the patent may see a cute kitten reaching for them. The patient needs to stretch to the desired distance in order to reach the kitten. This process may be created over and over again where the patient can stretch at their own pace. One advantage of a virtual kitten is that it does not run away or jump or move suddenly. Any desired object may be substituted for a kitten to encourage stretching.

### EXAMPLE 5

### Improving Neuroplasticity and Recovery—Audio Performance—Speech Therapy

In a prophetic example, a teacher records a tutorial with their voice saying words with specific tones, inflection, enunciating, and/or pronunciation. The system records data points for the frequency, tone, pitch, etc., of the voice and creates a wave visualizer of the voice. A patient/student in need of speech therapy from an injury, learning impairment, or other reason, selects the tutorial and is given step by step instructions to recreate the relevant parameters. The patient recreates each step and the system determines if the student is within the parameters. The student may receive a reward and then move to the next step in the tutorial. The present invention may allow for a patient to learn at their own pace and/or in privacy. The present invention is an improvement from the traditional videos and voice files that do not provide iterative feedback with unbiased, accurate, repeatable metrics.

### EXAMPLE 6

### Recreating a Picture

A student selects a tutorial for drawing a picture and downloads the tutorial to an iPad® after paying for the tutorial. The student sees the picture on their iPad and touches the image to get a preview of the picture as it was drawn. The student clicks begin and sees the first step of the tutorial, a line of the picture appears. This line displays on the screen in the same style and speed as originally drawn by the teacher. The student uses their finger or a stylus and draws over the line on the screen. The student may set the parameters of how exact they need to be in tracing/redrawing each step. If the step is not followed within the parameters, the system may prompt the student to repeat the step by erasing the line and starting from the beginning of the step.

The student may choose to have help or guidelines to help them draw/trace over the line. The student may also choose to hear the audio commentary of the teacher.

When the student completes the step within the parameters by tracing over the original drawing, the next step automatically starts and a new line appears on the screen in the same style and speed as originally drawn by the teacher. The student now completes the next step by tracing over the new line.

In one embodiment, the student has the choice to half draw over the line and then the original line will still stay on the screen until it has been fully traced over or has been traced over to within a percentage of the completed line. The tolerance of how far to draw over may be set by the teacher, student, health care professional if the student is a patient, etc.

In one embodiment, the student may also add different colors, styles, or different drawing tools to the artwork.

In one embodiment, students may choose to have guide tools to help and encourage them to complete the tutorial.

In one embodiment, when the student finishes a tutorial, they may choose to share the picture via social media or export it to be printed on a physical item like a cup, shirt, hat, etc., or have artwork bundled into a book. In an embodiment, students may choose to export a video of their tutorial to YouTube, Instagram, Facebook, etc.

### EXAMPLE 7

### Live Drawing a Picture

A teacher coordinates with one or more students for creating a drawing tutorial for drawing a picture in real time using the system of the present invention. The teacher may draw live from anywhere in the world and students follow along from anywhere in the world. The system records every line, cluster, color change, erasing, etc., that the teacher adds to the picture. The system may record audio of the teacher explaining parts of the tutorial. The system turns the recording into discreet sequential steps in chronological order and may create one or more tutorials for students to follow to recreate the picture. The system may allow for students to ask questions of the teacher, for example, via audio and/or text. The drawing and/or recreated drawing may also be converted into several forms for the purpose of sharing with others by the teacher and/or the students.

### EXAMPLE 8

### Audio Performance—Dramatic Reading

“To be, or not to be. That is the question.” In a prophetic example, as part of a drama class, a teacher records Hamlet's famous quote from Shakespeare. The system breaks the speech into steps. The teacher decides to make the first two sentences one step instead of two steps, and changes the program to merge these steps together.

A student repeats the speech, reading the lines for each step. The system determines if the reading is within current parameters set by the teacher when creating the tutorial. The tutorial may require emphasis to be placed on different words for different takes.

### EXAMPLE 9

### Teaching a Group of Animation Artists How to All Draw the Same Character and/or Objects to be Indistinguishable from Other Artists

Currently in the professional animation industry, if/when an animation show is crewing up for animators, they will often issue a “style guide” of all the characters/objects to all the animators on the show to study. The present invention may be used as a more efficient style guide. Prior art style guides are just a pdf or printed copy of static images that show what to do and what not to do when drawing the characters in the show. The goal of the style guide is so that many animators/artists can learn to draw the same characters indistinguishably from others. An example of a style guide is how to draw Homer Simpson, as many animators will be needed to draw Homer indistinguishably from other animators working on the show.

The present invention may have the original artist/teacher create a style guide inside the system and then the style guide may be distributed to the current and prospective artists involved. The system may be able to record which artist can most accurately redraw the cartoon or animated characters in the closest copy to the original artist. The system may also be able to suggest which artists are the best at recreating the style without the artistic team even needing to judge the artwork in person.

Style guides are static and therefore do not show the pace or direction of how the art was created. The present invention may show these features and this may aid in another artist to better learn how to draw the character in an accelerated way.

### EXAMPLE 10

### Combining Tutorials

In a prophetic example, a student selects a first tutorial from a first teacher, then selects a second tutorial from a second teacher and combines the tutorials. The system links the tutorials, creating a longer, more complex, and/or more detailed custom tutorial of their own making.

The custom tutorial promotes continued engagement of the muscle memory activity, which promotes neural plasticity. This increases the student's engagement when it is necessary for the student to continue with the activity for rehabilitation needs but the tasks and activities have become boring and/or too repetitive. Because the system allows for the student to add tutorial content from other tutorials, it is akin to combining LEGO® bricks from several different sets to create novel combinations.

### EXAMPLE 11

### Calligraphy

In a prophetic example, a student selects a calligraphy tutorial. Writing calligraphy and learning to write calligraphy may require several different movements of the stylus to create the desired effect. For example, the present invention allows a teacher to turn their stylus at a 45-degree angle and draw a line for 5 cm. Then, after the teacher has drawn along the indicated path, the system allows for another angle change and direction change of the stylus to be recorded. The present invention records the angles and paths that the stylus takes, and then, when the student engages in a tutorial, they see the exact path that the teacher took. The system will simulate the ink placement as if the student held a calligraphy brush.

### EXAMPLE 12

### Preserving Languages

There are numerous reports and articles about languages around the world that are fading and dying. Languages are passed down through spoken and written word. In some instances in North America, there are languages of first nation tribes in Canada and the USA where there may be only one surviving member who is a native speaker of the language.

Previously, many languages were recorded through video and audio and in some cases just written. But these previous methods are not as efficient as the present invention in also capturing a way to recreate and teach these languages to the next generation. They are more often categorized as a way to document the language.

In a prophetic example, the present invention may be used to have a native speaker write down the language, the alphabet, the symbols, and the characters. The present invention may be used to capture the exact and accurate way the language should be written and spoken.

The present invention may allow a single surviving member simply record their language knowledge into the present invention, and then, through the system, be able to teach generations of people interested in learning the language.

The present invention allows for a teacher to create tutorials of how to write and speak the language. The present invention is designed to have the teacher's tutorials to be distributed to interested people all over the world who have the same desire to learn that specific language.

Research states that one strong way to preserve a culture is to preserve the language and to teach it to the next generation. The present invention is a modern way to teach language learning that may be more stimulating to the new generation of students who may have short attention spans.

In some languages like Japanese and Chinese, it is also important for the language to be written down in a specific direction and order. Of the thousands of languages around the world, it may be that other languages that are not as well-known as Japanese and Chinese also have this learning requirement.

The present invention is able to recreate the correct direction of the stroke of the teacher in the tutorial so that a student may learn the correct stroke order.

The present invention may also be used to let the student import other objects from other tutorials and combine those object(s) into their current tutorial. For example, a student may want to learn to write two symbols in a sequence, but the first symbol was in one tutorial and the second symbol is in another tutorial. The system may allow the student to select the second tutorial and import the second symbol into the active tutorial.

It is to be understood that the foregoing description of embodiments has been presented for illustrative purposes and is not intended to limit the invention to the present embodiments. Other embodiments can be made without departing from the scope of the present invention. Accordingly, many modifications and variations are possible in light of the above teachings. It is therefore intended that the scope of the invention not be limited by specific examples in the detailed description.

All features disclosed in the specification, including the claims, abstract, and drawings, and all of the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise. Thus, unless expressly stated otherwise, each feature disclosed is one example only of a generic series of equivalent or similar features.

The foregoing detailed description of the present invention is provided for purposes of illustration, and it is not intended to be exhaustive or to limit the invention to the particular embodiments disclosed. The embodiments can provide different capabilities and benefits, depending on the configuration used to implement the key features of the invention. Accordingly, the scope of the invention is defined only by the following claims.

What is claimed is:

1. A system for rehabilitating a patient, the system comprising:
a computer;
a touch screen that is coupled to the computer; and
a stylus that is coupled to the computer and configured for use with the touch screen;
wherein:
stored within the computer is a tutorial that includes tutorial data points that when displayed on the touch screen represent an object that is subdividable into portions that are to be redrawn by the patient,
the tutorial includes a sequence of steps that are to be performed by the patient,
the computer is configured to parse the tutorial data points into a plurality of sets of tutorial data points,
each of the plurality of sets of tutorial data points corresponds to one of the portions of the object that is to be redrawn by the patient using the stylus in combination with the touch screen during one of the sequence of steps of the tutorial,
each of the plurality of sets of tutorial data points has associated with it at least one parameter,
the touch screen is configured to display for viewing by the patient a first portion of the object, the first portion of the object has associated with it a first set of tutorial data points,
after the first portion of the object is displayed on the touch screen, the computer prompts the patient to attempt to redraw using the stylus in combination with the touch screen the first portion of the object,
the computer automatically records patient data points collected from the patient's attempt to redraw the first portion of the object using the stylus in combination with the touch screen, and
the computer automatically compares the patient data points to the first set of tutorial data points, and when the computer determines that the patient data points are within the at least one parameter for the first set of tutorial data points, the computer automatically prompts the display on the touch screen of a second portion of the object for the patient to redraw, otherwise, if the patient data points are not within the at least one parameter for the first set of tutorial data points, the computer prompts the patient again to attempt to redraw the first portion of the object using the stylus in combination with the touch screen.

2. The system of claim 1, wherein:
the sequence of steps of the tutorial includes a start step and a finish step;
the computer automatically records the patient data points for each of the portions of the object from the start step of the tutorial until the finish step of the tutorial; and
the computer prompts the display on the touch screen the patient data points for each of the portions of the object.

3. The system of claim 1, wherein the patient data points include data selected from the group consisting of a pressure of the stylus, an angle of the stylus, and a time associated with use of the stylus.

4. The system of claim 1, wherein at least one of the portions of the object includes at least one line.

5. The system of claim 1, wherein the at least one parameter is adjustable.

6. The system of claim 1, wherein the step of the computer automatically recording the patient data points that are collected from the patient's attempt to redraw each of the portions of the object is repeated for each step of the tutorial until the tutorial is complete.

7. A method for rehabilitating a patient, the method comprising:
providing a computer;
providing a touch screen that is coupled to the computer;
providing a stylus that is coupled to the computer and configured for use with the touch screen;
providing a tutorial that is stored within the computer, the tutorial including tutorial data points that when displayed on the touch screen represent an object that is subdividable into portions that are to be redrawn by the patient, the tutorial includes a sequence of steps that are to be performed by the patient, and the computer is configured to parse the tutorial data points into a plurality of sets of tutorial data points with each of the plurality of sets of data points corresponding to one of the portions of the object that is to be redrawn by the patient using the stylus in combination with the touch screen during one of the sequence of steps of the tutorial, each of the plurality of sets of tutorial data has associated with it at least one parameter;
displaying for viewing by the patient a first portion of the object on the touch screen, the first portion of the object has associated with it a first set of tutorial data points;
prompting the patient to attempt to redraw using the stylus in combination with the touch screen the first portion of the object that is displayed on the touch screen;
automatically recording in the computer patient data points collected from the patient's attempt to redraw the first portion of the object using the stylus in combination with the touch screen; and
using the computer to automatically compare the patient data points to the first set of tutorial data points, and when the computer determines that the patient data points are within the at least one parameter for the first set of tutorial data points, using the computer to automatically prompt the display on the touch screen of a second portion of the object for the patient to redraw, otherwise, if the patient data points are not within the at least one parameter for the first set of tutorial data points, using the computer to prompt the patient again to attempt to redraw the first portion of the object using the stylus in combination with the touch screen.

8. The method of claim 7, wherein the sequence of steps of the tutorial includes a start step and a finish step, and the method further comprises:
using the computer to automatically record the patient data points for each of the portions of the object from the start step of the tutorial until the finish step of the tutorial; and
using the computer to prompt the display on the touch screen the patient data points for each of the portions of the object.

9. The method of claim 7, wherein the patient data points include data selected from the group consisting of a pressure of the stylus, an angle of the stylus, and a time associated with use of the stylus.

10. The method of claim 7, wherein at least one portion of the object includes at least one line.

11. The method of claim 7, wherein the at least one parameter is adjustable.

12. The method of claim 7, wherein the step of automatically recording the patient data points that are collected from the patient's attempt to redraw each of the portions of the object is repeated for each step of the tutorial until the tutorial is complete.

13. A system for rehabilitating a patient, the system comprising:
a computer;
a touch screen that is coupled to the computer; and
a stylus that is coupled to the computer and configured for use with the touch screen;
wherein:
stored within the computer is a tutorial that is configured to prompt the patient to redraw an object that is displayed on the touch screen using the stylus in combination with the touch screen,
the object is subdivided into a plurality of portions,
the tutorial includes a sequence of steps that are to be performed by the patient,
in each of the steps of the sequence of steps, the patient is prompted to redraw at least one portion of the object,
each of the plurality of portions of the image has associated with it at least one parameter,
the touch screen is configured to display for viewing by the patient a first portion of the object,
after the first portion of the object is displayed on the touch screen, the computer prompts the patient to attempt to redraw using the stylus in combination with the touch screen the first portion of the object,
the computer automatically records data collected from the patient's attempt to redraw the first portion of the object using the stylus in combination with the touch screen, and
the computer automatically compares the data to the first portion of the object, and when the computer determines that the data are within the at least one parameter for the first portion of the object, the computer automatically prompts the display on the touch screen of a second portion of the object for the patient to redraw,
otherwise, if the data are not within the at least one parameter for the first portion of the object, the computer prompts the patient again to attempt to redraw the first portion of the object using the stylus in combination with the touch screen.

14. The system of claim 13, wherein:
the sequence of steps of the tutorial includes a start step and a finish step;
the computer automatically records the data from the patient's attempts to redraw each of the portions of the object from the start step of the tutorial until the finish step of the tutorial; and
the computer prompts the display on the touch screen the data for each of the portions of the object.

15. The system of claim 13, wherein the data is selected from the group consisting of a pressure of the stylus, an angle of the stylus, and a time associated with use of the stylus.

16. The system of claim 13, wherein at least one of the portions of the object includes at least one line.

17. The system of claim 13, wherein the at least one parameter is adjustable.

18. The system of claim 13, wherein the step of the computer automatically recording the data that are collected from the patient's attempt to redraw each of the portions of the object is repeated for each step of the tutorial until the tutorial is complete.
